(19) United States
(12) Patent Application Publication
(10) Pub. No.: US 2004/0225375 A1
(43) Pub. Date: Nov. 11, 2004

(54) Prosthetic foot having a universal joint in an ankle member

(76) Inventor: Sen-Jung Chen, Taipei City (TW)

Correspondence Address: LADAS & PARRY LLP, 224 SOUTH MICHIGAN AVENUE, SUITE 1200, CHICAGO, IL, 60604, US

(21) Appl. No.: 10/431,759

(22) Filed: May 8, 2003

(51) Int. Cl. A61F 2/62; A61F 2/66

(52) U.S. Cl. 623/38; 623/49

(57) ABSTRACT

A prosthetic foot includes a foot body having heel and toe parts, an ankle member disposed above a joint hole in the heel part and having two opposite ends and an intermediate part, an ankle shaft having a bottom end formed with a ball below the intermediate part, a socket receiving the ball to form a ball-and-socket joint, two spring members mounted within the heel part below the ankle member and respectively adjacent to the opposite ends so as to support the ankle member, and a retaining unit fixed to the heel part within the joint hole for retaining the socket within the joint hole.

### BACKGROUND OF THE INVENTION

1. Field of the Invention

The invention relates to a prosthetic foot, more particularly to a prosthetic foot having an ankle member provided with a ball-and-socket joint.

2. Description of the Related Art

Referring to FIG. 1, a conventional prosthetic foot includes a foot body 1, a retaining unit 2, an ankle member 3, and a horizontal shaft 4 interconnecting pivotally the retaining unit 2 and the ankle member 3. The retaining unit 2 has a lower threaded shank portion 201, an upper ring portion 202 connected fixedly to a top portion of the shank portion 201, a ball bearing 203 disposed in the ring portion 202, and two supporting pads 204 disposed on each side of the ring portion 202 so as to center the retaining unit 2 relative to the ankle member 3. The ball bearing 203 includes a ring seat 2031 mounted fittingly in the ring portion 202, and a single ball 2032 disposed rotatably in the ring seat 2031. The shaft 4 extends through the ankle member 3, the ball 2032, and the supporting pads 204.

Although the conventional prosthetic foot described above is provided with a universal joint, since the shaft 4 that interconnects the retaining unit 2 and the ankle member 3 is in a horizontal direction, the retaining unit 2 has to be centered to provide good balance for the user. However, the centering of the retaining unit 2 requires additional component parts. Furthermore, the foot body 1 has to provide a receiving space that is relatively large so as to receive the lower part of the retaining unit 2 which covers the horizontal shaft 4 and the additional components.

### SUMMARY OF THE INVENTION

Therefore, the object of the present invention is to provide a prosthetic foot that is capable of overcoming the aforementioned drawbacks of the prior art.

According to this invention, a prosthetic foot comprises a foot body having a heel part and a toe part, the heel part having a joint hole, an elongated ankle member disposed above the joint hole and having two longitudinally opposite ends and an intermediate part between the opposite ends, an ankle shaft extending through the intermediate part of the ankle member and having a bottom end formed with a ball below the intermediate part, a socket receiving the ball to form a ball-and-socket joint, two spring members mounted within the heel part below the ankle member and respectively adjacent to the opposite ends so as to support the ankle member, and a retaining unit fixed to the heel part within the joint hole for retaining the socket within the joint hole.

### BRIEF DESCRIPTION OF THE DRAWINGS

Other features and advantages of the present invention will become apparent in the following detailed description of the preferred embodiments with reference to the accompanying drawings, of which:

FIG. 1 is an exploded perspective view of a conventional prosthetic foot;

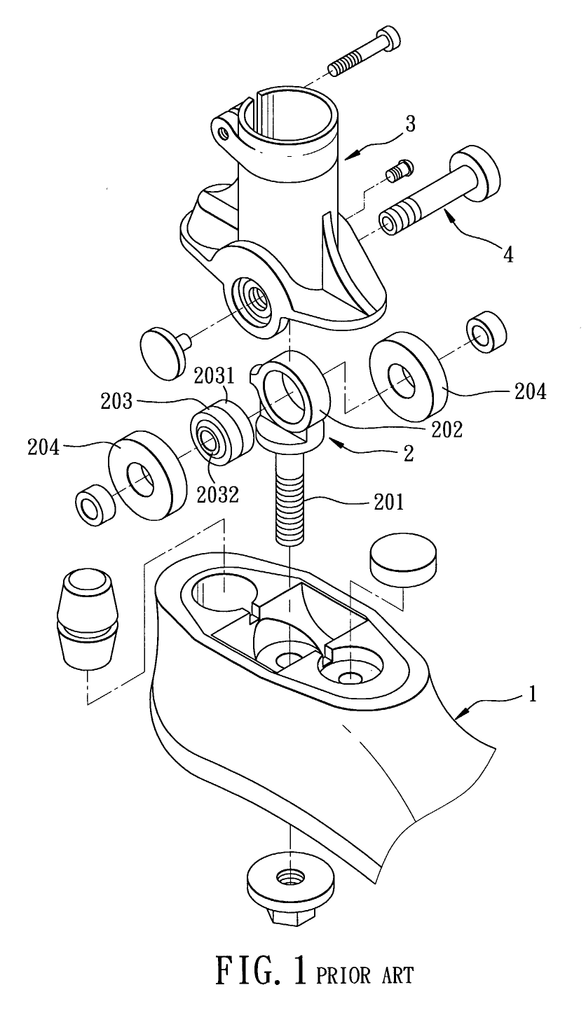

FIG. 2 is an exploded perspective view of the first preferred embodiment of a prosthetic foot according to the present invention;

FIG. 3 is a sectional view of the first preferred embodiment in an assembled state;

FIG. 4 is the same view as FIG. 3, but with the heel part of the foot body being pressed against the ground surface;

FIG. 5 is an exploded perspective view of the second preferred embodiment of a prosthetic foot according to the present invention; and

FIG. 6 is a sectional view of the second preferred embodiment in an assembled state.

### DETAILED DESCRIPTION OF THE PREFERRED EMBODIMENTS

Before the present invention is described in greater detail, it should be noted that like elements are denoted by the same reference numerals throughout the disclosure.

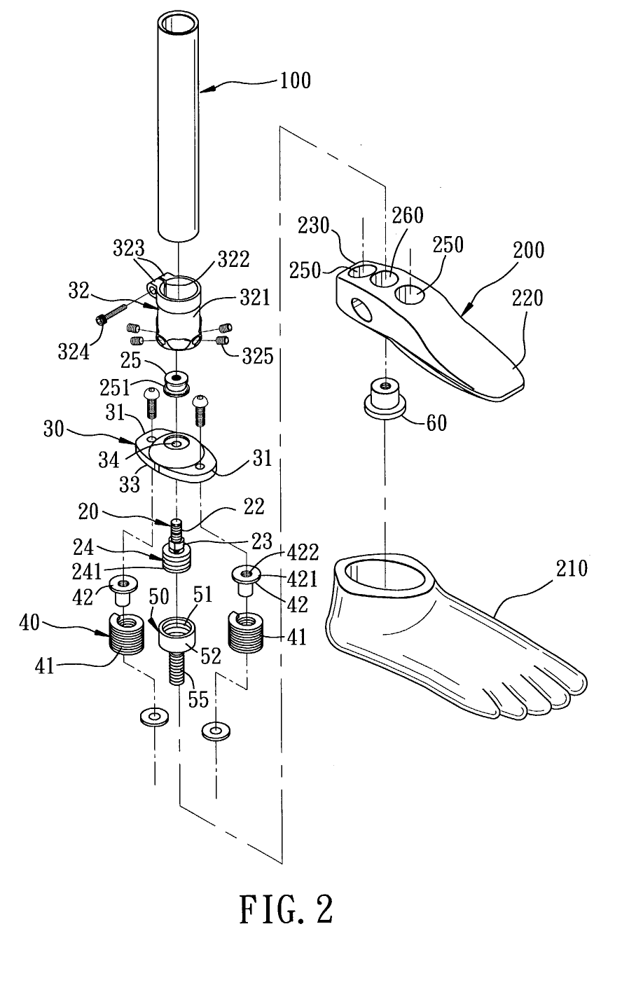

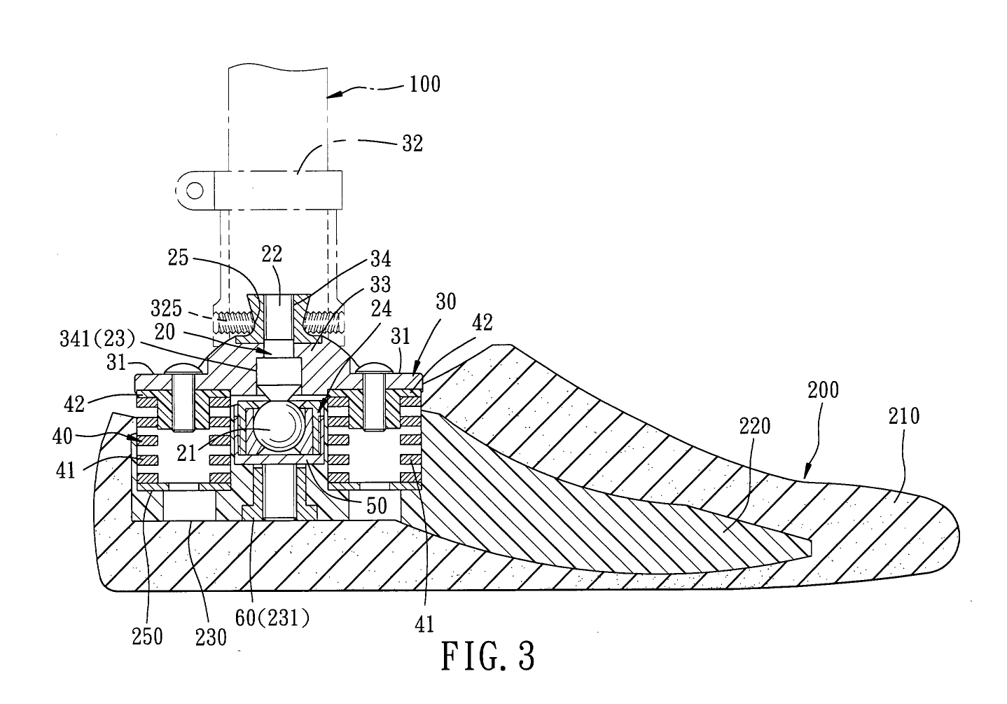

Referring to FIGS. 2 and 3, the first preferred embodiment of a prosthetic foot according to the present invention is adapted to be connected to a leg member 100, and is shown to comprise a foot body 200, an ankle shaft 20, a socket 24, an elongated ankle member 30, two spring members 40, and a retaining unit 50.

The foot body 200 is inserted into a unitary molded shell body 210 made of a flexible foam material, and has a toe part 220 and a heel part 230. The heel part 230 has a joint hole 260, and two spring holes 250 on two sides of the joint hole 260.

The ankle shaft 20 has a bottom end formed with a ball 21 (see FIG. 3), and includes a screw portion 22 opposite to the ball 21, a positioning part 23 between the screw portion 22 and the ball 21, and a nut 25 attached to the screw portion 22. The positioning part 23 has a multi-sided cross section. The nut 25 has an outer periphery formed with an annular groove 251.

The socket 24 is attached to the ankle shaft 20 by engaging the socket 24 with the ball 21. Thus, the socket 24 receives the ball 21 to form a ball-and-socket joint.

The elongated ankle member 30 is disposed above the joint hole 260 in the heel part 230, and has two longitudinally opposite ends 31, an intermediate part 33 between the opposite ends 31 and having a dome-shaped top surface, and a through hole 34 formed in the intermediate part 33 for passage of the ankle shaft 20 therethrough. The ankle shaft 20 extends through the through hole 34 in such a manner that the screw portion 22 and the ball 21 of the ankle shaft 20 are located respectively above and below the intermediate part 33 of the ankle member 30. The nut 25 engages the screw portion 22 to lock the ankle member 30 relative to the ankle shaft 20. The through hole 34 has a part 341 (see FIG. 3) for receiving the positioning part 23, and has a cross-section substantially corresponding to the multi-sided cross section of the positioning part 23. The part 341 of the through hole 34 receives the positioning part 23 so as to prevent relative rotational movement between the ankle shaft 20 and the ankle member 30.

The prosthetic foot further includes a shin 32. The shin 32 includes a tubular wall 321 extending around the screw portion 22 and the nut 25 and having an open end seated on top of the dome-shaped top surface of the intermediate part 33 of the ankle member 30, and a plurality of clamp screws 325 extending radially and threadedly through the tubular wall 321 and having ends engaging the annular groove 251 in the nut 25. The tubular wall 321 has a split 322, two clamping lugs 323 integrally formed with the tubular wall 321 adjacent to two sides of the split 322, and a clamping screw 324 extending threadedly through the clamping lugs 323 so that the tubular wall 321 can be clamped tightly against the leg member 100.

The spring members 40 are mounted within the heel part 230 below the ankle member 30 and are respectively adjacent to the opposite ends 31 of the ankle member 30 so as to support the latter. In this embodiment, each of the spring members 40 is a spring coil 41. Each of the spring coils 41 is received in a corresponding one of the spring holes 250 in the heel part 230 of the foot body 200, and incorporates a hollow screw seat 42 snugly fitted into the corresponding spring coil 41. Each screw seat 42 has a top flange 421 seated on top of the corresponding spring coil 41, and a female screw 422 formed inside the screw seat 42. The ankle member 30 is screwed to the screw seat 42 through the female screw 422.

The retaining unit 50 is fixed to the heel part 230 within the joint hole 260 for retaining the socket 24 within the joint hole 260, and includes a receiving space 51 and a retaining wall 52 confining the receiving space 51. The socket 24 has a socket wall with an outer wall face 241 engaging threadedly the retaining wall 52. The retaining unit 50 is screwed to the heel part 230 by means of a screw rod 55 which is formed integrally with the retaining wall 52 and which extends downwardly to threadedly engage a female screw member 60. The female screw member 60 is received in a bottom hole 231 formed at the bottom of the heel part 230.

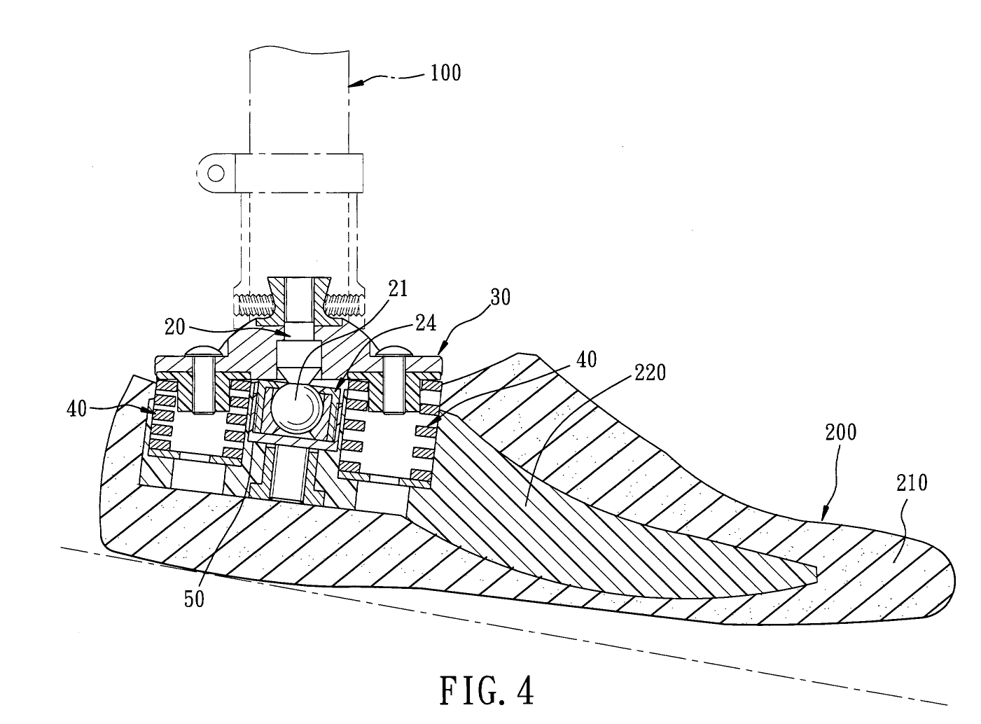

When the user, after wearing the prosthetic foot of the present invention, strides during walking, a heel part of the shell body 210 initially presses against the ground surface so that the rear one of the spring members 40 absorbs the shock and deforms, as shown in FIG. 4. Afterwards, the entire bottom face of the shell body 210 contacts the ground surface, and the deformed spring member 40 is restored to a normal state shown in FIG. 3.

Due to the configuration of the retaining unit 50 that engages threadedly the socket 24, and the vertical ankle shaft 20 that is integral with a ball-and-socket joint, the prosthetic foot of the present invention is simple and easy to assemble and repair. In addition, the vertical ankle shaft 20 can be centered with respect to the shin 32 using fewer components as compared to that of the conventional prosthetic foot.

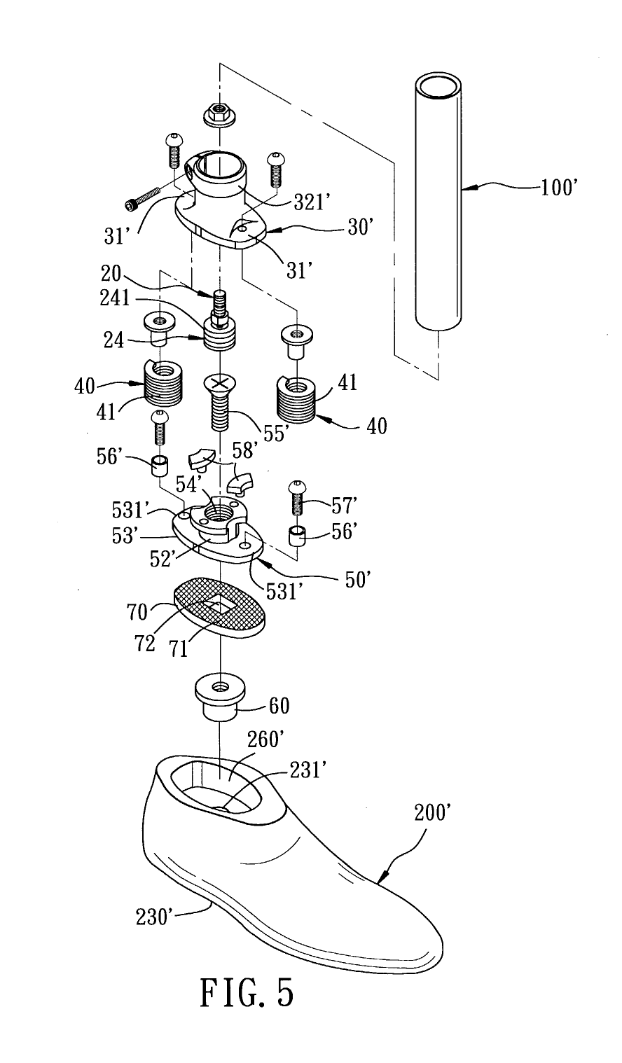

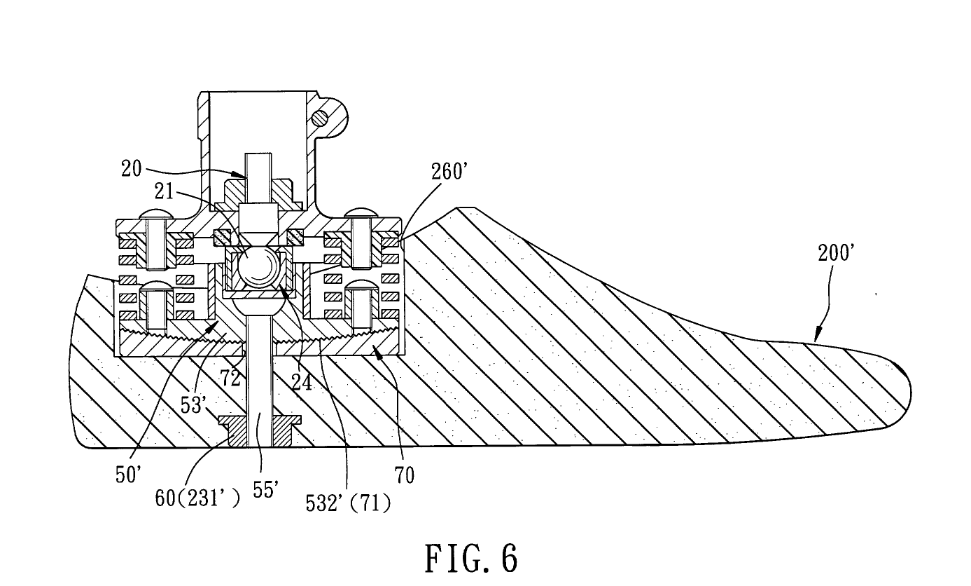

Referring to FIGS. 5 and 6, the second preferred embodiment of a prosthetic foot according to the present invention is shown to be substantially similar to the first preferred embodiment. However, in this embodiment, the ankle member 30′ further includes a tubular wall 321′ extending upward therefrom between the opposite ends 31′ to serve as a shin. The ball 21 (See FIG. 3) of the ankle shaft 20, the socket 24, and the spring members 40 are received in a single joint hole 260′ in the foot body 200′. The retaining unit 50′ includes an elongated base plate 53′ having two longitudinally opposite ends 531′ and received in the joint hole 260′ below the socket 24 and the spring members 40. A retaining wall 52′ projects upward from the base plate 53′ between the opposite ends 531′ of the base plate 53′, and confines a receiving space 54′ to receive the ball 21 and the socket 24. The retaining wall 52′ surrounds the socket 24. The socket 24 has a socket wall with an outer wall face 241 threadedly engaging the retaining wall 52′.

The second preferred embodiment further comprises an adjustment plate 70 disposed in the joint hole 260′ and in contact with a bottom surface of the base plate 53′. The adjustment plate 70 has a top surface formed with a plurality of first flutes 71 (see FIG. 6), and a through hole 72.

The base plate 53′ of the retaining unit 50′ is screwed to the heel part 230′ by means of a screw rod 55′ that extends through the receiving space 54′ in the base plate 53′, the through hole 72 in the adjustment plate 70, and to the bottom of the heel part 230′ to engage a nut 60 within a bottom hole 231′ in the heel part 230′. The bottom surface of the base plate 53′ is formed with a plurality of second flutes 532′ (see FIG. 6) to engage the first flutes 71. The single joint hole 260′ has a cross-section greater than the cross-sections of the base plate 53′ and the adjustment plate 70, and the hole 72 in the adjustment plate 70 has a cross-section larger than that of the screw rod 55′. Therefore, the base plate 53′ is movable in the joint hole 260′ for position adjustment relative to the adjustment plate 70 according to the weight and the body configuration of the user. The first and second flutes 71, 532′ are releasably interengageable to position the base plate 53′ relative to the adjustment plate 70, and include intersecting flutes, which define protrusions thereamong.

The retaining unit 50′ further includes a pair of posts 56′ projecting upward from the base plate 53′ on two sides of the retaining wall 52′ to extend respectively into the spring coils 41 of the spring members 40. Each of the posts 56′ is formed as a hollow piece, and has a screw element 57′ which extends through the corresponding hollow piece and which is screwed to the base plate 53′. Two fillers 58′ are disposed on two opposite ends of the retaining wall 52′.

The advantages of the first preferred embodiment can be similarly attained using the second preferred embodiment of the present invention.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

I claim:

1. A prosthetic foot comprising:
a foot body having a heel part and a toe part, said heel part having a joint hole;
an elongated ankle member disposed above said joint hole and having two longitudinally opposite ends and an intermediate part between said opposite ends;
an ankle shaft extending through said intermediate part of said ankle member and having a bottom end formed with a ball below said intermediate part;
a socket receiving said ball to form a ball-and-socket joint;
two spring members mounted within said heel part below said ankle member and respectively adjacent to said opposite ends so as to support said ankle member; and
a retaining unit fixed to said heel part within said joint hole for retaining said socket within said joint hole.

2. The prosthetic foot as claimed in claim 1, wherein each of said spring members is a spring coil which incorporates a hollow screw seat fitted into said spring coil, said screw seat having a top flange seated on top of said spring coil and a female screw formed inside said screw seat, said ankle member being screwed to said screw seat through said female screw.

3. The prosthetic foot as claimed in claim 2, wherein said foot body further has two spring holes on two sides of said joint hole, said spring coil of each of said spring members being received in a corresponding one of said spring holes.

4. The prosthetic foot as claimed in claim 1, wherein said retaining unit includes a receiving space and a retaining wall confining said receiving space, said socket having a socket wall with an outer wall face engaging threadedly said retaining wall.

5. The prosthetic foot as claimed in claim 4, wherein said retaining unit is screwed to said heel part.

6. The prosthetic foot as claimed in claim 1, wherein said ankle shaft further includes a screw portion above said ankle member and a nut attached to said screw portion for locking said ankle member relative to said ankle shaft.

7. The prosthetic foot as claimed claim 6, wherein said ankle shaft further includes a positioning part between said screw portion and said ball, said positioning part having a multi-sided cross section, said ankle member further including a through hole for passage of said ankle shaft, said through hole having a part for receiving said positioning part and having a cross-section substantially corresponding to said multi-sided cross section, said part of said through hole receiving said positioning part so as to prevent relative rotational movement between said ankle shaft and said ankle member.

8. The prosthetic foot as claimed in claim 7, wherein said nut includes an outer periphery formed with an annular groove.

9. The prosthetic foot as claimed in claim 8, further comprising a shin which includes a tubular wall extending around said screw portion and said nut, and a plurality of clamp screws extending radially and threadedly through said tubular wall and having ends engaging said annular groove.

10. The prosthetic foot as claimed in claim 9, wherein said intermediate part of said ankle member has a dome-shaped top surface, said tubular wall having an open end seated on top of said dome-shaped top surface.

11. The prosthetic foot as claimed in claim 1, wherein said ankle member further includes a tubular wall extending upward therefrom between said opposite ends to serve as a shin.

12. The prosthetic foot as claimed in claim 2, wherein said ball, said socket and said spring members are received in said joint hole.

13. The prosthetic foot as claimed in claim 12, wherein said retaining unit includes an elongated base plate having two longitudinally opposite ends and received in said joint hole below said socket and said spring members, a retaining wall projecting upward from said base plate between said opposite ends of said base plate, and a receiving space confined by said retaining wall to receive said ball and said socket, said retaining wall surrounding said socket, said socket having a socket wall threadedly engaging said retaining wall.

14. The prosthetic foot as claimed in claim 13, wherein said base plate is screwed to said heel part.

15. The prosthetic foot as claimed in claim 14, wherein said retaining unit further includes a pair of posts projecting upward from said base plate on two sides of said retaining wall to extend respectively into said spring coils of said spring members.

16. The prosthetic foot as claimed in claim 14, wherein each of said posts is formed as a hollow piece and has a screw element which extends through said hollow piece and which is screwed to said base plate.

17. The prosthetic foot as claimed in claim 14, further comprising an adjustment plate disposed in said joint hole and in contact with a bottom surface of said base plate, said adjustment plate having a top surface formed with a plurality of first flutes, said bottom surface of said base plate being formed with a plurality of second flutes to engage said first flutes, said base plate being movable on said adjustment plate for position adjustment relative to said adjustment plate, said first and second flutes being releasably interengageable to position said base plate relative to said adjustment plate.

18. The prosthetic foot as claimed in claim 17, wherein said first and second flutes include intersecting flutes, which define protrusions thereamong.
